(19) United States
(12) Patent Application Publication
(10) Pub. No.: US 2016/0327623 A1
(43) Pub. Date: Nov. 10, 2016

(54) MAGNETIC RESONANCE IMAGING APPARATUS AND FAT SUPPRESSION WATER IMAGE CALCULATION METHOD

(71) Applicant: HITACHI, LTD., Tokyo (JP)

(72) Inventor: Norimasa NAKAI, Tokyo (JP)

(21) Appl. No.: 15/109,759

(22) PCT Filed: Jan. 15, 2015
(86) PCT No.: PCT/JP2015/050901

§ 371 Date: Jul. 5, 2016

(30) Foreign Application Priority Data
Jan. 30, 2014 (JP) 2014-015368

(51) Int. Cl. G01R 33/48; G01R 33/565; G01R 33/56; A61B 5/055; A61B 5/00

(52) U.S. Cl. CPC G01R 33/4828; A61B 5/055; A61B 5/4872; A61B 5/4875; G01R 33/5607; G01R 33/56545

(57) ABSTRACT

To obtain a water image in which a fat signal is suppressed by a desired ratio without damaging contrast by a simple method, the water image in which a fat signal remains by a desired ratio is obtained with high precision by weighting and adding a plurality of images obtained by reconstructing echo signals acquired at a plurality of different echo times. At this time, the plurality of different echo times are set so that a phase difference between water and fat signals included in the images is different in at least two images. A weight coefficient used for weighting and adding is decided so that a difference in a signal strength by a difference in the echo time is cancelled and the fat signal is suppressed by the desired ratio in the water image.

### TECHNICAL FIELD

The present invention relates to a nuclear magnetic resonance imaging (MRI) apparatus that measures nuclear magnetic resonance (NMR) signals from hydrogen, phosphorus, or the like inside an object and images a nuclear density distribution or an alleviation time distribution, and particularly, to a technology for acquiring a water image in which a fat signal is suppressed by a desired ratio.

### BACKGROUND ART

An MRI apparatus is an apparatus that measures NMR signals which are generated by nuclear spins forming an object, particularly, tissues of a human body and images forms or functions of a human head, abdomen, or limbs two-dimensionally or three-dimensionally. In the imaging, different-phase encoding is applied and frequency encoding is performed on the NMR signals by a gradient magnetic field, so that the NMR signals are measured as time-series data. The measured NMR signals are subjected to two-dimensional or three-dimensional Fourier conversion to reconstruct an image.

In a case in which an image is obtained with an MRI apparatus, an image having various tissue contrasts can be obtained by changing a parameter such as an echo time (TE) or a repetition time (TR) or performing image calculation. In clinical practice, an image in which signals from a fat tissue (fat signal) is suppressed is requested in some cases. As an example of a method of obtaining an image in which a fat signal is suppressed, there is a method of acquiring a plurality of images in which a TE is different and separating images (water images) reconstructed from signals (water signals) from water from images (fat images) reconstructed from fat signals through calculation. As a representative method, there is a method called a Dixon method.

In the Dixon method, a water image is obtained by adding an image obtained at an out-phase and an image obtained at an in-phase as in formula (1) below. Here, In indicates an image obtained at the in-phase, Out indicates an image obtained at the out-phase, W indicates an image (water image) in which each pixel is configured of a water signal, and F indicates an image (fat image) in which each pixel is configured of a fat signal.

W=Out+In=W−F+W+F (1)

where

In=W+F

Out=W−F

In recent years, an image indicating a content of fat created from a water image and a fat image is used in clinical practice. Such images are obtained by a plurality of images in which TE is different, separating water images from fat images through calculation, and mathematically combining the images (for example, see PTL 1).

### CITATION LIST

### Patent Literature

PTL 1: U.S. Pat. No. 7,592,810

### SUMMARY OF INVENTION

### Technical Problem

In clinical practice, an image in which a fat signal is suppressed is requested, but fat signals are desired to remain depending on an imaged target or an imaging type. For example, in imaging of knee, it is easy to comprehend a positional relation between tissues in image reading when bone signals slightly remain. In imaging of contrast called a T1 weighted image, fat signals are almost unnecessary since water signals are relatively large. However, in imaging of contrast called a T2 weighted image, fat signals preferably remain slightly since water signals are very small.

By adding fat signals to water signals at a desired ratio after separating water signals (water image) and fat signals (fat signals) by Formula (1), the fat signals can remain in the water image. In this method, however, since water images and fat images are once separated from a plurality of images of different TEs and the images are further recombined, a calculation time may prolong. Further, since generated images are increased, a use amount of a memory in which a calculator is necessary is increased.

In the Dixon method, since there is an influence of T2*attenuation in signals between different echoes, a difference occurs between echo signals. Due to the difference between the echo signals, a fat signal slightly remains in a water image in some cases when water and fat images are separated.

For example, when a TE with an out-phase is shorter than a TE with the in-phase in a gradient sequence and a fat signal with the in-phase is smaller by 10% than a fat signal with an out-phase due to the influence of T2 and T2* attenuation, a negative fat signal remains in the water image obtained by adding the in-phase and the out-phase as in Formula (2) below. In Formula (2) below, the influence of the T2 and T2* attenuation of the water signal is assumed to be the same as that of the fat signal.

W=Out+0.9×In=W−F+0.9×(W+F)=1.9W−0.1F (2)

Unlike an intentionally remaining signal, the phase of a remaining fat signal is different from the phase of a water signal of a water image in some cases, and thus correct contrast may be damaged. In this case, the contrast of a recombined image may be damaged.

The present invention is devised in view of the foregoing circumstances and an object of the present invention is to provide a technology for obtaining a water image in which a fat signal remains by a desired ratio with high precision by a simple method without damaging contrast and without calculating a separate image in which water and fat signals are separated.

### Solution to Problem

According to the present invention, a water image in which a fat signal is suppressed by a desired ratio is obtained by weighting and adding a plurality of images obtained by reconstructing echo signals acquired at a plurality of different echo times. At this time, in the plurality of different echo times, a phase difference between water and fat signals included in images is set differently in at least two images. A weight coefficient used for weighting addition is decided so that a difference in a fat signal strength by a difference in an echo time is cancelled and a fat signal is suppressed by the desired ratio in a water image.

### Advantageous Effects of Invention

According to the present invention, it is possible to obtain a water image in which fat signals remain by a desired ratio with high precision by a simple method without damaging contrast and without calculating a separate image in which water and fat signals are separated.

### BRIEF DESCRIPTION OF DRAWINGS

FIG. 1 is a diagram illustrating the entire configuration of a magnetic resonance imaging apparatus according to an embodiment of the present invention.

FIG. 2 is an explanatory diagram illustrating a gradient echo (GE) sequence of a two-point Dixon method.

FIG. 3 is a diagram illustrating the configuration of a signal processing unit according to the embodiment of the present invention.

FIGS. 4(a) and 4(b) are explanatory diagrams illustrating a fat suppression coefficient input region according to the embodiment of the present invention and FIG. 4(c) is an explanatory diagram illustrating a fat ratio table according to the embodiment of the present invention.

FIG. 5 is a flowchart illustrating a fat suppression image generation process according to the embodiment of the present invention.

FIGS. 6(a) to 6(c) are explanatory diagrams illustrating an example of a fat suppression image by simulation.

FIGS. 7(a) and 7(b) are explanatory diagrams illustrating an effect of signal strength correction by a difference in a TE according to the embodiment of the present invention.

FIG. 8 is a flowchart illustrating a fat suppression image generation process according to a modification example of the embodiment of the present invention.

### DESCRIPTION OF EMBODIMENTS

An embodiment of the present invention will be described with reference to the appended drawings. The same reference numerals are given to constituents having the same functions unless specified otherwise throughout all the drawings for describing the embodiment of the present invention, and the repeated description thereof will be omitted.

### <Configuration of MRI Apparatus>

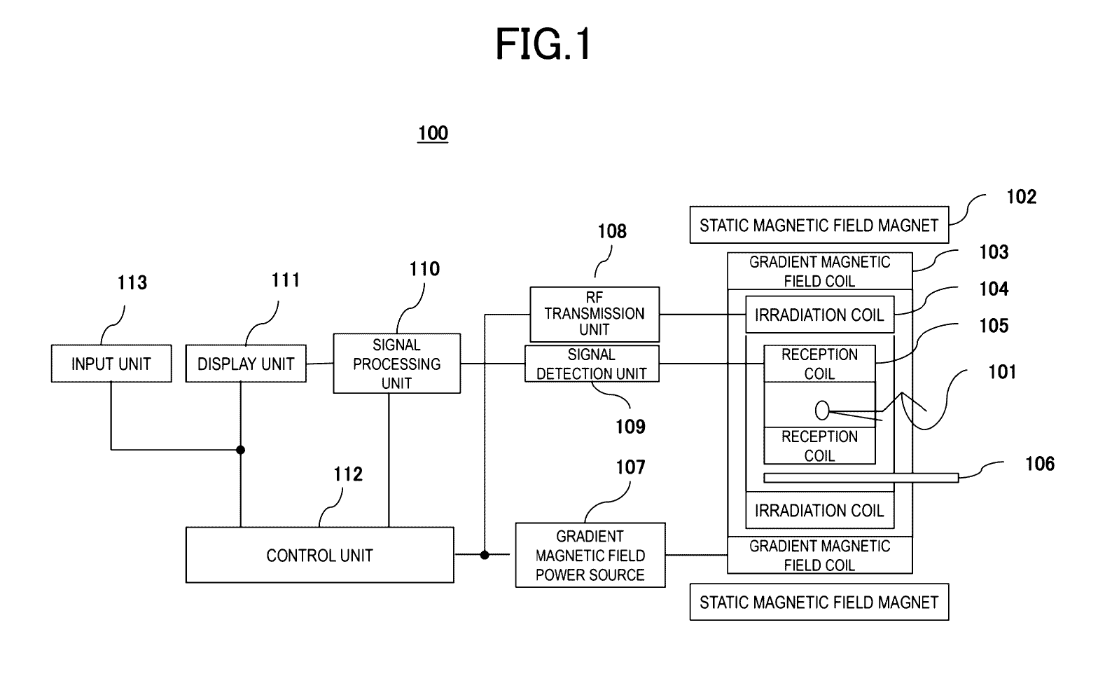

The configuration of an example of an MRI apparatus according to the embodiment will be described. FIG. 1 is a functional block diagram illustrating an MRI apparatus 100.

The MRI apparatus 100 according to the embodiment includes static magnetic field generation magnets 102, gradient magnetic field coils 103, irradiation coils 104, reception coils 105, and a bed 106 on which an object 101 is put transversely, a gradient magnetic field power source 107, an RF transmission unit 108, a signal detection unit 109, a signal processing unit 110, a display unit 111, a control unit 112, and an input unit 113.

The static magnetic field generation magnet 102 generates a uniform static magnetic field in a direction parallel or vertical to a body axis around the object 101. The static magnetic field generation magnet 102 is configured of any one of a permanent magnet, a super-conductive magnet, and a normal conductive magnet disposed in a space having a predetermined expanse around the object 101.

The gradient magnetic field coils 103 apply gradient magnetic fields in tri-axis directions of X, Y, and Z to the object 101 according to signals from the gradient magnetic field power source 107. In accordance with a method of applying the gradient magnetic fields, imaged cross sections of an object are set.

The irradiation coils 104 generate high-frequency magnetic field pulses (RF pulses) according to signals from the RF transmission unit 108. Nuclei of atoms forming biological tissues of the imaged cross sections are excited by the RF pulses, and thus an NMR phenomenon is caused.

An echo signal which is an NMR signal generated by the NMR phenomenon is detected by the signal detection unit 109 via the reception coils 105 disposed near the object 101 and is converted into an image through signal processing by the signal processing unit 110.

The display unit 111 displays the image converted by the signal processing unit 110. An interface screen of an input of the input unit 113 is displayed as necessary.

The input unit 113 receives an input of a parameter from an operator. The input parameter is, for example, a repetition time (TR) or an echo time (TE) necessary for imaging. The input parameter is transmitted to the display unit 111 to be displayed. Similarly, such a parameter is transmitted to the control unit 112.

The control unit 112 controls the gradient magnetic field power source 107, the RF transmission unit 108, and the signal processing unit 110 according to the parameter received from the input unit 113. These units are controlled to repeatedly generate a slice encoding gradient magnetic field, a phase encoding gradient magnetic field, a frequency encoding gradient magnetic field, and the RF pulses according to a predetermined pulse sequence.

The control unit 112 and the signal processing unit 110 include a CPU, a memory, and a storage device. These functions are realized when the CPU loads a program stored in the storage device to the memory and executes the program.

In the embodiment, as in the two-point Dixon method, echo signals are each acquired at a TE (TE1: first echo time) at which the phases of water and fat signals are reversed and a TE (TE2: second echo time) at which the phases of the water and fat signals are the same. Then, the image is reconstructed from the respective echo signals to obtain the image.

Hereinafter, the image reconstructed from the echo signals acquired at the TE1 is referred to as an image at the time of the out-phase and the image reconstructed from the echo signals acquired at the TE2 is referred to as an image at the time of the in-phase. In the embodiment, the TE1 is assumed to be less than the TE2.

In the embodiment, a water image in which the fat signal is suppressed by a desired ratio is obtained from the image at the time of the out-phase and the image at the time of the in-phase without separating the water and fat images. Further, at this time, in the embodiment, an influence of T2 and T2* attenuation by a time difference between the first and second echo times is also corrected. Hereinafter, a pulse sequence and a process of the signal processing unit for realizing the correction will be described.

### <Pulse Sequence>

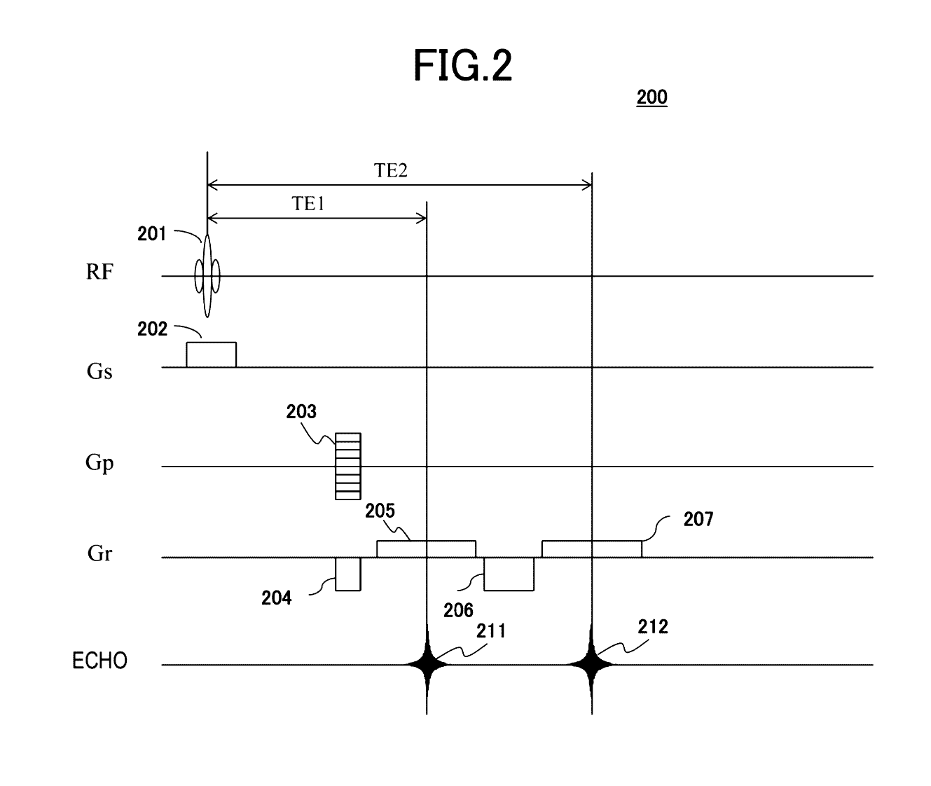

An example of a pulse sequence used in the two-point Dixon method and the embodiment will be described based on a sequence chart illustrated in FIG. 2. A pulse sequence 200 is a sequence of a gradient echo (GE) sequence method and two types of images in which the TE is different are obtained.

In the embodiment, as described above, images are obtained at the TE1 and the TE2.

A slice encoding gradient magnetic field 202 is applied simultaneously with radiation of an RF pulse 201. Accordingly, only target tomographic planes are excited. A phase encoding gradient magnetic field 203 for encoding positional information is applied and a frequency encoding gradient magnetic field (pre-pulse) 204 in a negative direction is applied simultaneously. Thereafter, a frequency encoding gradient magnetic field 205 in a positive direction is applied to generate a first echo signal 211 from the RF pulse 201 after the TE1 has passed.

Next, after a frequency encoding gradient magnetic field (rewind-pulse) 206 in a negative direction is applied again, a frequency encoding gradient magnetic field 207 is applied to generate a second echo signal 212 from the RF pulse after the TE2 has passed.

By repeatedly performing the sequence by the number of times of the phase encoding while changing the area of the phase encoding gradient magnetic field 203, echo signals corresponding to the number of phase encoding are acquired to be filled in a k space.

The foregoing pulse sequence is realized when the control unit 112 controls operations of the gradient magnetic field power source 107, the RF transmission unit 108, and the signal detection unit 109.

### <Signal Processing Unit>

Next, the details of the signal processing unit 110 that processes the obtained echo signal will be described. In the embodiment, the signal processing unit 110 performs two-dimensional Fourier conversion on data of the k space to obtain two types of images in which the TE is different. That is, a first image (out-phase image) is obtained from the data of the k space in which the first echo signal is filled and a second image (in-phase image) is obtained from the data of the k space in which the second echo signal is filled. A desired image is obtained from such two images.

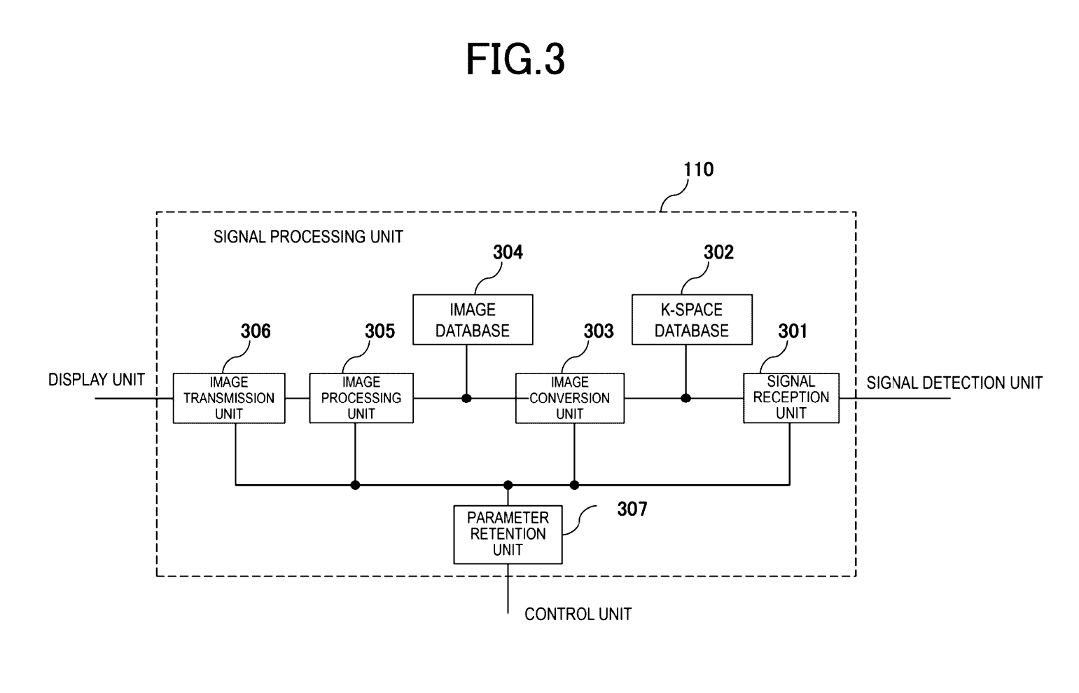

FIG. 3 is a functional block diagram illustrating the signal processing unit 110. As illustrated in the drawing, the signal processing unit 110 according to the embodiment includes a signal reception unit 301, a k-space database 302, an image conversion unit 303, an image database 304, an image processing unit 305, an image transmission unit 306, and a parameter retention unit 307.

The signal reception unit 301 stores an echo signal detected by the signal detection unit 109 in the k-space database 302 based on disposition information in the k space retained by the parameter retention unit 307.

The disposition in the k space is specified by slice encoding, frequency encoding, and phase encoding. In the embodiment, a different k space is prepared for each TE, and each echo signal is stored.

The image conversion unit 303 performs Fourier conversion on the data of each k space stored in the k-space database 302 to reconstruct each image and stores the image in the image database 304. In the embodiment, an out-phase image and an in-phase image are obtained.

The image processing unit 305 performs image processing on each image stored in the image database 304 and delivers the processed image to the image transmission unit 306. Examples of the image processing include processes of correcting unevenness of sensitivity of the reception coil 105. In the embodiment, a fat suppression image generation process of calculating a water image in which a fat signal is suppressed by the desired ratio is performed by weighting and adding the out-phase image and the in-phase image. The details of the fat suppression image generation process will be described later.

The image transmission unit 306 transmits the image subjected to the image processing to the display unit 111.

The parameter retention unit 307 retains the disposition information in the k space required by the signal reception unit 301, that is, information regarding the slice encoding, the frequency encoding, and the phase encoding of the pulse sequence, or control information and parameters of image matrix or filtering required by the image conversion unit 303, the image processing unit 305, and the image transmission unit 306. Such parameters are acquired from the control unit 112.

In the embodiment, the parameter retention unit 307 also retains attenuation correction coefficients for correcting a difference in a fat signal strength by T2 and T2* attenuation and parameters (fat suppression coefficients) for designating a ratio by which a fat signal is suppressed to use them at the time of the fat suppression image generation process. The attenuation correction coefficient and the fat suppression coefficient will be described later.

### <Fat Suppression Image Generation Process>

In the embodiment, the image processing unit 305 obtains the water image in which the fat signal is suppressed by the desired ratio without separating the water and fat images, from the out-phase image and the in-phase image in the fat suppression image generation process. At this time, a difference in the fat signal strength by T2 and T2* attenuation between the signals by the time difference in the TE is corrected.

Specifically, a weight coefficient to be multiplied to each of the out-phase image and the in-phase image is decided using an attenuation correction coefficient to be multiplied to correct the difference in the fat signal strength or a signal strength attenuation coefficient which is a reciprocal of the attenuation correction coefficient and indicates a difference in the signal strength and a fat suppression coefficient for specifying a ratio by which the fat signal is suppressed. That is, the weight coefficient to be multiplied to each of the out-phase image and the in-phase image is decided so that the difference in the fat strength by T2 and T2* attenuation between signals by a difference in an acquisition timing is corrected and the fat signal is suppressed according to the designated ratio by which the fat signal is suppressed.

For example, the fat suppression coefficient, that is, the ratio by which the fat signal is suppressed is assumed to be (1−α) (0≦α≦1). Here, α indicates a fat remaining rate. That is, the fat signals are assumed to remain in a water image by the ratio of α. Further, β_{F} is assumed to be an attenuation correction coefficient for correcting the influence of T2 and T2* attenuation of the fat signals by an acquisition timing.

In this case, weight coefficients A and B applied to an out-phase image Out and an in-phase image In are calculated as in Formula (3) below.

W_{supF}=A×Out+B×In (3)
where
   A=(1−α)×β_{F}
   B=(1+α)

Here, W_{supF} is a water image in which the fat signal is suppressed by the desired ratio.

When Formula (3) is modified, Formula (4) below is obtained. It can be understood that W_{supF} has the fat signal remained by the ratio of a and the water signal. In Formula (4), γ_{W} is a ratio of an out-phase to an in-phase in regard to signal attenuation of a water signal by the influence of T2 and T2* attenuation and γ_{F} is a ratio of an out-phase to an in-phase in regard to signal attenuation of a fat signal by the influence of T2 and T2* attenuation. Hereinafter, γ_{W} and γ_{F} are referred to as attenuation coefficients. Here, the attenuation correction coefficient β_{F} is assumed to be set such that the fat signal strength of the out-phase is the same as the fat signal strength of the in-phase. That is, the attenuation correction coefficient β_{F} is a reciprocal (β_{F}=1/γ_{F}) of the attenuation coefficient γ_{F}. At this time, since β_{F}γ_{W} is near 1, a water signal has about 2 W.

$$\begin{matrix}{W}_{supF} = ( 1 - α ) \times {β}_{F} \times Out + ( 1 + α ) \times In \\ = ( 1 - α ) \times {β}_{F} ( {γ}_{W} W - {γ}_{F} F ) + ( 1 + α ) \times ( W + F ) \\ = ( 1 + {β}_{F} {γ}_{W} ) W + ( 1 - {β}_{F} {γ}_{W} ) α W + 2 α F\end{matrix} \qquad ( 4 )$$

In Formulae (3) and (4) above, an influence of a phase by non-uniformity of a static magnetic field is assumed to be removed by correction.

The weight coefficients are not limited to A and B in Formula (3) above. The weight coefficients may be obtained by Formulae (5) and (6) below in which the coefficient of one of the out-phase image and the in-phase image is set to 1.

W_{supF}=A₁×Out+In

A₁=(1−α)/(1+α)×β_{F} (5)

W_{supF}=Out+B₁×In

B₁=(1+α)/((1−α)×β_{F}) (6)

The weight coefficient is decided so that a ratio of the signal strength of the out-phase to the signal strength of the in-phase is increased as a ratio by which a fat signal remains in a water image is larger.

### <Fat Suppression Coefficient>

The fat suppression coefficient (1−α) is input via the input unit 113 from a user and is retained in the parameter retention unit 307. In this case, the control unit 112 according to the embodiment includes an interface that receives designation of a fat suppression coefficient from the user. For example, the interface is assumed to display a fat suppression coefficient input region 810 on the display unit 11 and this region is assumed to be input via the input unit 113 by the user.

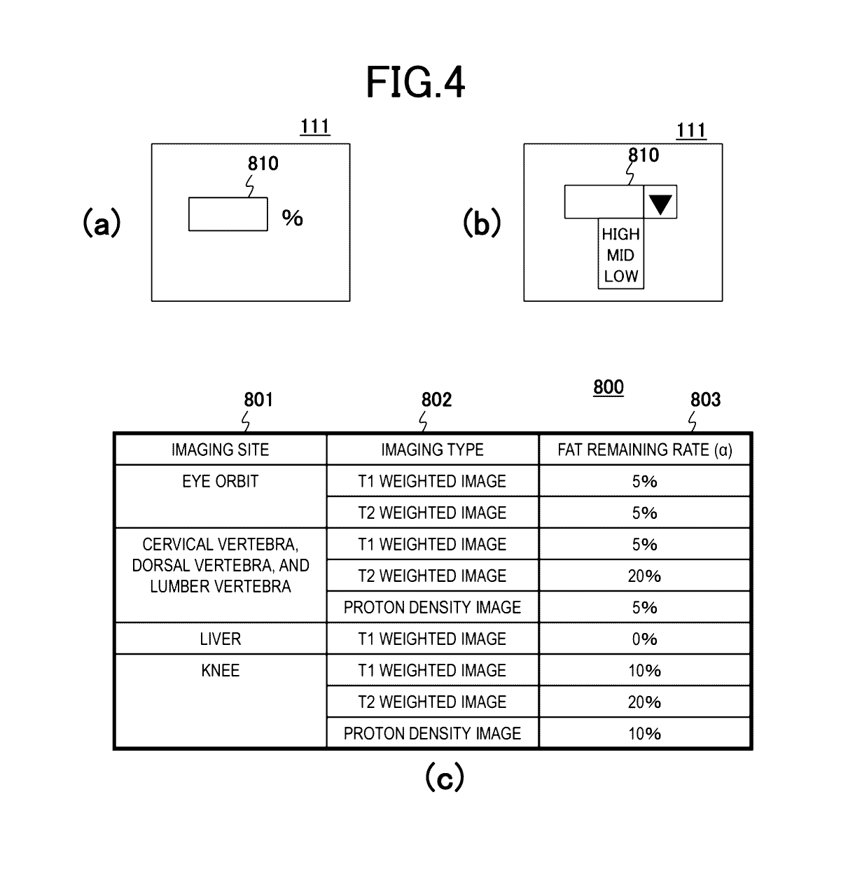

For example, the input of the fat suppression coefficient may be configured as an input of a percentage ratio by which fat is suppressed via the fat suppression coefficient input region 810 displayed on the display unit 111, as illustrated in FIG. 4(a). As illustrated in FIG. 4(b), the ratio may be selected from preset values displayed in the fat suppression coefficient input region 810.

For example, in a case of a percentage input scheme, 95% is input. Upon receiving this, the image processing unit 305 performs control such that only 5% of fat remains.

On the other hand, in a method of selecting the ratio from preset values, for example, items “strong”, “intermediate”, and “weak” are prepared as degrees of fat suppression and one of the items is configured to be selectable. Suppression coefficient values are associated with the items to be retained. For example, a suppression coefficient 100% is associated with the item “strong”, 90% is associated with the item “intermediate”, and 80% is associated with the item “weak”. That is, when the user selects the item “strong”, the image processing unit 305 performs control such that 0% of fat remains. When the user selects the item “intermediate”, the image processing unit 305 performs control such that 10% of fat remains. When the user selects the item “weak”, the image processing unit 305 performs control such that 20% of fat remains.

Further, the user may not designate the ratio of the fat suppression, but may conversely designate a ratio by which fat is desired to remain. In this case, in a case in which 5% of fat is desired to remain, 5% is input. Upon receiving this, the image processing unit 305 performs control such that 5% of fat remains.

As the ratio by which fat remains, there is a ratio appropriate according to an imaging site and an imaging type. Accordingly, optimum ratios may be retained in advance as a fat ratio table according to imaging sites and imaging type in the parameter retention unit 307 and may be adopted automatically according to selection of imaging sites and imaging type.

That is, the control unit 112 has a fat ratio table as a database to retain a ratio in which the fat signal is suppressed in association with at least one of an imaging site and an imaging type. Then, the image processing unit 305 acquires the ratio from the database according to an imaging site or an imaging type set by the user.

In this case, a parameter of information regarding the imaging site or the imaging type is transmitted from the input unit 113 to the control unit 112, the parameter a of the fat ratio is selected from a relation between the fat ratio and the imaging site or the imaging type stored in advance in the control unit 112, the parameter a is transmitted to the signal processing unit 110, and the parameter a is used by the signal processing unit 110.

FIG. 4(c) illustrates an example of a fat ratio table 800 in which an appropriate fat suppression coefficient is retained for each imaging site 801 and each imaging type 802. Here, a case in which a fat remaining rate a in a water image from which the fat suppression coefficient can be calculated is retained will be exemplified. The image processing unit 305 calculates the fat suppression coefficient (1−α) from the fat remaining rate a and uses the fat suppression coefficient (1−α) to calculate a weight coefficient.

In imaging of an eye orbit, it is necessary to suppress fat of the eye orbit wholly. Therefore, the fat remaining rate a is set to be small as 5% in both of a T1 weighted image and a T2 weighted image.

In imaging of cervical vertebra, dorsal vertebra, and lumber vertebra, a water signal is relatively large in a T1 weighted image and a proton density image. Therefore, the fat remaining rate a is set to be small as 5%. Since a water signal is small in a T2 weighted image, the fat remaining rate a is set to 20% to easily comprehend a tissue or the like.

In imaging of liver, it is necessary to calculate a fat ratio quantitatively from output water and fat images. Therefore, it is proper to set the fat remaining rate a to 0% so that the fat does not remain.

A water signal is relatively large in a T1 weighted image and a proton density image of a knee, but it is necessary to remain a bone signal, and therefore the fat remaining rate a is set to 10%. Since a water signal is small in a T2 weighted image and it is necessary to remain a bone signal, it is proper to set the fat remaining rate a to 20%.

The suppression coefficient input via the input unit 113 is transmitted to the signal processing unit 110 via the control unit 112 and is used when a water image in which a fat signal is suppressed by a desired ratio is generated by the signal processing unit 110. The fat ratio table 800 may be stored in advance in the signal processing unit 110.

### <Attenuation Correction Coefficient>

The attenuation correction coefficient β_{F} is a coefficient which is applied to one image between an in-phase image and an out-phase image to correct the influence of T2 and T2* attenuation. The attenuation correction coefficient is decided so that no fat signal with an out-phase remains in a finally obtained water image. In the embodiment, the attenuation correction coefficient is decided so that a fat signal strength with an out-phase is equal to a fat signal strength with an in-phase.

For example, as in the example of Formula (2) above, in a case in which a fat pixel value (fat signal) of an image with an in-phase is 0.9 times a fat pixel value (fat signal) of an image with an out-phase, the attenuation correction coefficient is set to 0.9 in Formulae (4) to (6) above.

The attenuation correction coefficient β_{F} is decided by a type of sequence and the TEs of the in-phase and the out-phase. Accordingly, the attenuation correction coefficient β_{F} is decided in advance based on actually measured values acquired by changing the TE in various sequences, is associated with the sequence species and the TEs, and is retained as, for example, a correction coefficient database in the parameter retention unit 307. In a case in which the influence of T2 and T2* attenuation is small or a case in which high precision is not required, β_{F} may be neglected as 1.

### <Flow of Fat Suppression Image Generation Process>

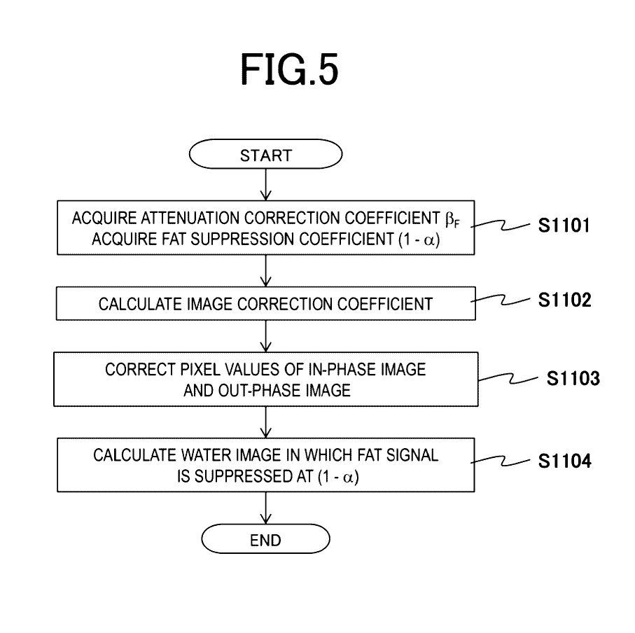

FIG. 5 is a flowchart illustrating the fat suppression image generation process performed by the image processing unit 305 according to the embodiment. As described above, the present process is stored as a program in a storage device and the image processing unit 305 performs a process of each step.

(step S1101) The attenuation correction coefficient β_{F} of T2 and T2* between an image (out-phase image) reconstructed from an echo signal acquired at the first echo time TE1 and an image (in-phase image) reconstructed from an echo signal acquired at the second echo time TE2 is acquired from the parameter retention unit 307. The fat signal suppression ratio (1−α) designated by the user is acquired.

(step S1102) The weight coefficient to be applied to each of the out-phase image and the in-phase image is calculated using the attenuation correction coefficient β_{F} and the fat signal suppression ratio (1−α). Here, for example, one of A or B of Formula (3) above, A₁ of Formula (5) above, and B₁ of Formula (6) above is calculated. Such coefficients are calculated once in imaging in which one image is obtained.

(step S1103) Pixel values of the in-phase image and the out-phase image are corrected using the weight coefficient calculated in step S1102. This calculation is performed by the number of image pixels and the number of imaged slices.

(step S1104) The water image in which the fat signal is suppressed by the desired ratio (1−α) is obtained by adding the corrected in-phase image and out-phase image.

Hereinafter, simulation results according to the embodiment will be described.

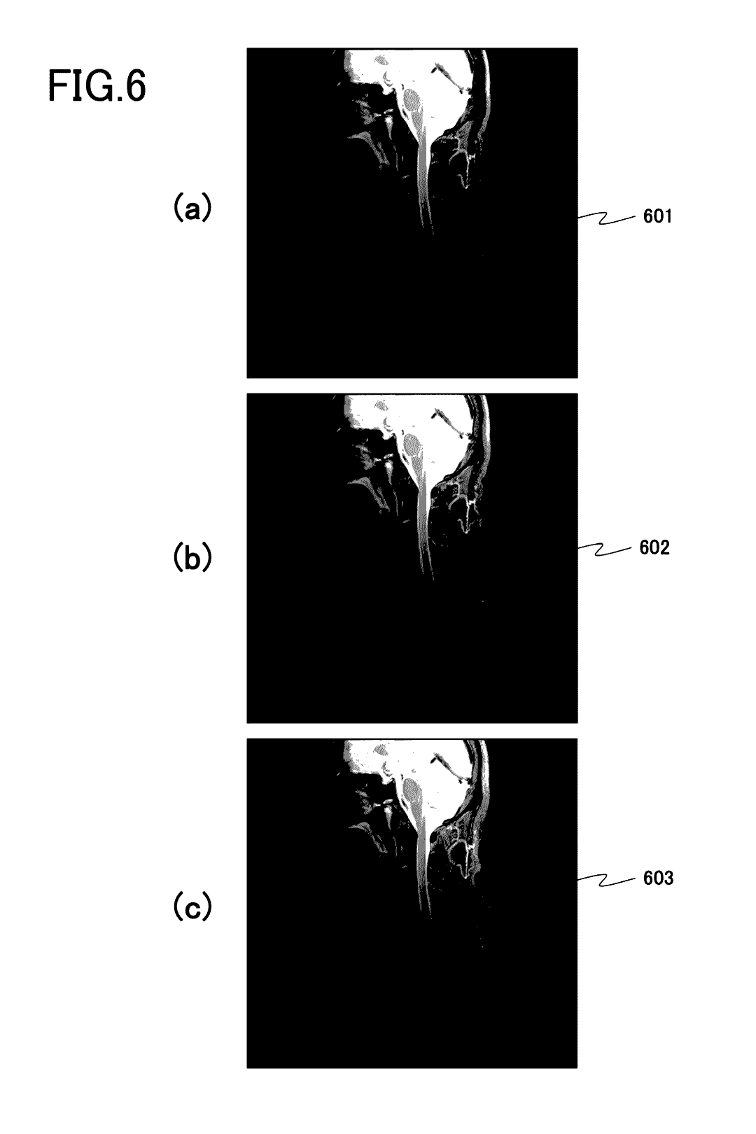

FIGS. 6(a) to 6(c) illustrate water images (T2 weighted images) obtained without performing the signal strength correction by the difference in the TE using the out-phase image and the in-phase image. An image 601 in FIG. 6(a) is an image in which the ratio α of the fat signal remaining in the water image is set to 0, an image 602 in FIG. 6(b) is an image in which the ratio α of the fat signal remaining in the water image is set to 10%, and an image 603 in FIG. 6(c) is an image in which the ratio α of the fat signal remaining in the water image is set to 20%.

By increasing the ratio α of the fat signal, it can be understood that the fat signal is appropriately increased over a back from an occipital region.

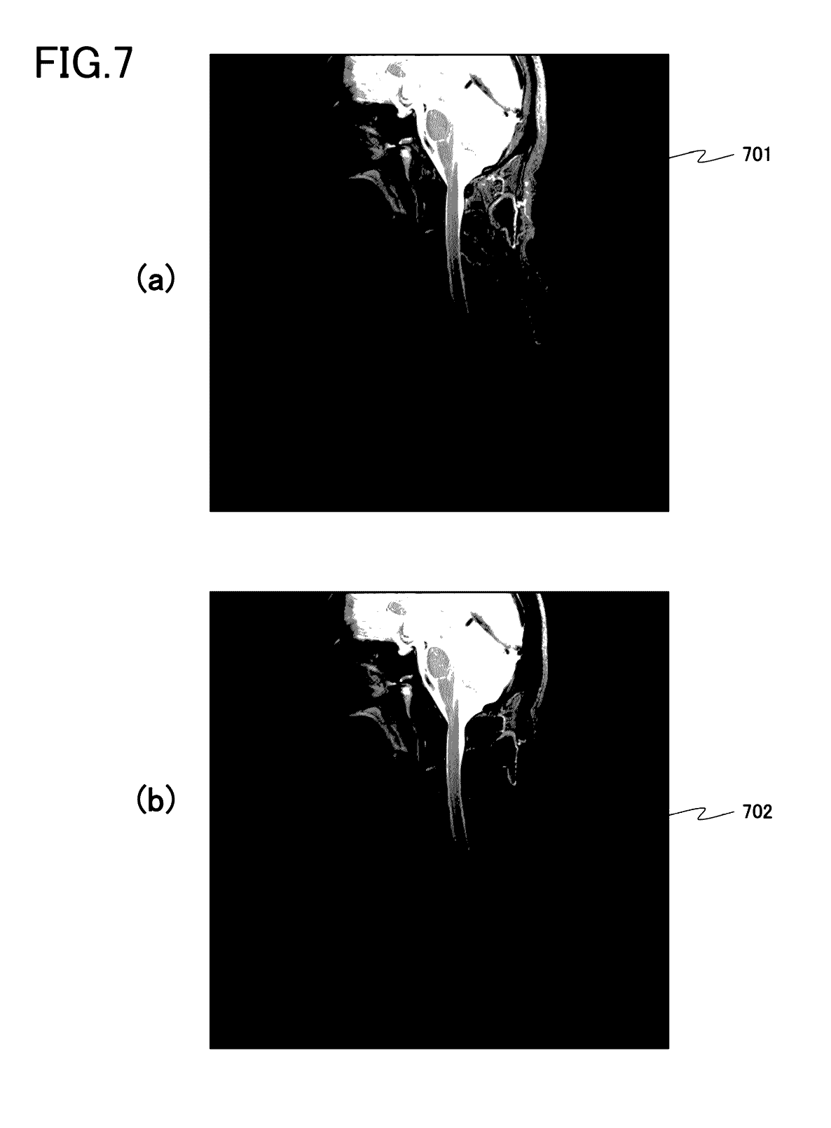

FIGS. 7 (a) and 7 (b) are diagrams for describing an effect of the scheme according to the embodiment in which the signal strength correction is performed by the difference in the TE using the out-phase image and the in-phase image. An image 701 in FIG. 7 (a) is an image in which the attenuation correction coefficient β is set so that the signal strength of the in-phase image is greater than the signal strength of the out-phase image and 20% of fat remains and an image 702 in FIG. 7 (b) is an image in which the attenuation correction coefficient β is set so that the signal strength of the out-phase image is greater than the signal strength of the in-phase image and 20% of fat remains.

In the image 701, good quality contrast is maintained and the fat signal remains. However, in the image 702, since the phase of the fat signal is oriented to be opposite to the water signal, the water signal is cancelled and the contrast is damaged.

In this way, in a case in which the signals are acquired at the out-phase and the in-phase, the phase of the fat signal remaining in the water image can be arranged with the phase of the water signal by setting the fat signal with the out-phase to be equal to or less than the signal with the in-phase.

Accordingly, it is possible to generate the water image in which the fat signal remains at a high speed without damaging the contrast.

### Modification Example 1

In the foregoing embodiment, two different TEs have been configured as the TE1 at which the fat and water signals are at the out-phase and the TE2 at which the fat and water signals are at the in-phase as in normal two-point Dixon method, but the two TEs are not limited thereto. A phase difference between the water and fat signals of the first image reconstructed from the echo signal acquired at the first echo time may be different from a phase difference between the water and fat signals of the second image reconstructed from the echo signal acquired at the second echo time.

In a case in which the influence of non-uniformity of a static magnetic field may not be considered, the phase of the fat signal of the first image reconstructed from the echo signal acquired at the first echo time may be different from the phase of the fat signal of the second image reconstructed from the echo signal acquired at the second echo time. Further, the phase of the water signal and the phase of the fat signal may be different in at least one of the first and second images.

In a case in which an image of two or more echoes is obtained at an echo time (TE) at which the forgoing conditions are satisfied, simultaneous equations in which a water image in which the fat signal remains by any ratio is an unknown are generated using a signal of an image acquired at each TE, a phase rotation amount by a fat chemical shift at each TE, and the attenuation coefficient (the reciprocal of the attenuation correction coefficient) of T2 and T2* attenuation. The weight coefficients A and B are obtained by solving the simultaneous equations.

Hereinafter, a method of calculating the weight coefficients A and B to be multiplied to an image acquired at each TE in a case in which two images are acquired at the TEs will be described.

Hereinafter, any two echo times satisfying the foregoing conditions are set as a first echo time TE1 and a second echo time TE2 (where TE1<TE2). An image reconstructed from an echo signal acquired at the first echo time is referred to as a first image and an image reconstructed from an echo signal acquired at the second echo time is referred to as a second image.

A signal S1 of the first image and a signal S2 of the second image are each expressed by Formula (7) below. Here, the influence of the phase by the non-uniformity of a static magnetic field is assumed to be removed by correction.

S₁=W+Fexp(iθ₁)

S₂=γ_{W}W+γ_{F}Fexp(iθ₂) (7)

Here, θ₁ is a phase by fat chemical shift at the time TE1, θ₂ is a phase by fat chemical shift at the time TE2, γ_{W} is an attenuation coefficient of the signal strength of the second image to the first image by the T2 and T2* attenuation of a water signal, and γ_{F} is the same attenuation coefficient of the fat signal.

By solving the simultaneous equations of Formula (7), water and fat images can be obtained.

By expressing Formula (7) as a matrix, Formula (8) below is obtained.

$$[ \begin{matrix}{S}_{1} \\ {S}_{2}\end{matrix} ] = [ \begin{matrix}1 & exp ( i {θ}_{1} ) \\ {γ}_{W} & {γ}_{F} exp ( i {θ}_{2} )\end{matrix} ] [ \begin{matrix}W \\ F\end{matrix} ] \qquad ( 8 )$$

By solving the formula, the water and fat images are obtained as in Formula (9) below.

$$[ \begin{matrix}W \\ F\end{matrix} ] = {[ \begin{matrix}1 & exp ( i {θ}_{1} ) \\ {γ}_{W} & {γ}_{F} exp ( i {θ}_{2} )\end{matrix} ]}^{- 1} [ \begin{matrix}{S}_{1} \\ {S}_{2}\end{matrix} ] \qquad ( 9 )$$

Here, [ ]⁻¹ indicates an inverse matrix.

In a case in which the fat signal remains in the water image by a ratio α (0≦α≦1), Formula (7) above is converted into Formal (10) below.

S₁=W+αF+Fexp(iθ₁)−αF

S₂=γ_{W}W+γ_{W}αF+γ_{F}Fexp(iθ₂)−γ_{W}αF (10)

In Formula (10), positive and negative αF terms are added to the right side of S₁ in Formula (7) and a γ_{W}αF term is added to S₂. Since the positive and negative αF terms and the γ_{W}αF term are mutually cancelled, this formula is equivalent to Formula (7).

The water image in which the fat signal remains by α (0≦α≦1), that is, the water image in which the fat signal is suppressed by (1−α), is expressed as W+αF. Accordingly, W+αF is obtained by solving the simultaneous equations of Formula (10) for W+αF and F.

When Formula (10) is expressed as a determinant, Formula (11) below is obtained.

$$[ \begin{matrix}{S}_{1} \\ {S}_{2}\end{matrix} ] = [ \begin{matrix}1 & exp ( i {θ}_{1} ) - α \\ {γ}_{W} & {γ}_{F} exp ( i {θ}_{2} ) - {γ}_{W} α\end{matrix} ] [ \begin{matrix}W + α F \\ F\end{matrix} ] \qquad ( 11 )$$

By solving the determinant, W+α is obtained as in Formula (12) below. According to the calculation, a fat image F is also obtained simultaneously.

$$[ \begin{matrix}W + α F \\ F\end{matrix} ] = [ \begin{matrix}1 & exp ( i {θ}_{1} ) - α \\ {γ}_{W} & {γ}_{F} exp ( i {θ}_{2} ) - {γ}_{W} α\end{matrix} ] [ \begin{matrix}{S}_{1} \\ {S}_{2}\end{matrix} ] \qquad ( 12 )$$

At this time, coefficients related to S1 and S2 are the foregoing weight coefficients A and B.

### Modification Example 2

Further, in the embodiment, a water image in which a fat signal is suppressed by a desired ratio may be obtained by weighting and adding images acquired at three or more different echo times. In this case, at the echo times, a phase difference between water and fat signals included in the images may be set to be different in at least two images.

Hereinafter, a method of calculating the weight coefficients to be multiplied to an image acquired at each TE in a case in which images are acquired at the TEs will be described. Even in this case, as described above, W+αF is obtained by solving simultaneous equations for W+αF and F.

In a case in which n echo signals are acquired at n different (where n is an integer equal to or greater than 3) TEs, a signal Sₙ of an image reconstructed from an echo signal acquired at each TE can be expressed in the following formula. Hereinafter, an image reconstructed from an n-th echo signal is referred to as an n-th image.

$${S}_{1} = W + F exp ( i {θ}_{1} ) \\ {S}_{2} = {γ}_{2 W} W + {γ}_{2 F} F exp ( i {θ}_{2} ) \\ … \\ {S}_{n} = {γ}_{nW} W + {γ}_{n F} F exp ( i {θ}_{n} ) \qquad ( 13 )$$

Here, γ_{nW} is an attenuation coefficient by the influence of T2 and T2* attenuation of a water signal in an n-th image by a water signal in the first image and γ_{nF} is an attenuation coefficient by the influence of T2 and T2* attenuation of a fat signal in the n-th image with respect to a fat signal in the first image.

In a case in which the fat signal remains in the water image by a ratio α (0≦α≦1), Formula (13) above is converted into Formal (14) below.

$${S}_{1} = W + α F + F exp ( i {θ}_{1} ) - α F \\ {S}_{2} = {γ}_{2 W} W + {γ}_{2 W} α F + {γ}_{2 F} F exp ( i {θ}_{2} ) - {γ}_{2 W} α F \\ … \\ {S}_{n} = {γ}_{nW} W + {γ}_{nW} α F + {γ}_{2 F} F exp ( i {θ}_{n} ) - {γ}_{nW} α F \qquad ( 14 )$$

When Formula (14) is expressed as a matrix, Formula (15) below is obtained.

$$[ \begin{matrix}{S}_{1} \\ {S}_{2} \\ ⋮ \\ {S}_{n}\end{matrix} ] = [ \begin{matrix}1 & exp ( i {θ}_{1} ) - α \\ {γ}_{2 W} & {γ}_{2 F} exp ( i {θ}_{2} ) - {γ}_{2 W} α \\ ⋮ & ⋮ \\ {γ}_{nW} & {γ}_{n F} exp ( i {θ}_{2} ) - {γ}_{nW} α\end{matrix} ] [ \begin{matrix}W + α F \\ F\end{matrix} ] \qquad ( 15 )$$

When each component is put as in Formula (16) below, Formula (15) can be expressed as Formula (17) below.

$$\begin{matrix}[ \begin{matrix}{S}_{1} \\ {S}_{2} \\ ⋮ \\ {S}_{n}\end{matrix} ] = S , [ \begin{matrix}1 & exp ( i {θ}_{1} ) - α \\ {γ}_{2 W} & {γ}_{2 F} exp ( i {θ}_{2} ) - {γ}_{2 W} α \\ ⋮ & ⋮ \\ {γ}_{nW} & {γ}_{n F} exp ( i {θ}_{2} ) - {γ}_{nW} α\end{matrix} ] = C , [ \begin{matrix}W + α F \\ F\end{matrix} ] = P & ( 16 ) \\ S = C \cdot P & ( 17 )\end{matrix}$$

Formula (17) is solved for a vector P as in Formula (18) below to obtain each component of P, that is, the water image in which the fat signal remains by a is obtained.

S=C·P

C^{H}·S=C^{H}C·P

P=(C^{H}C)⁻¹C^{H}·S (18)

Here, ^{H} indicates an adjoint matrix.

At this time, a coefficient related to each Sₙ is the weight coefficient.

Even in the embodiment, the attenuation coefficients γ_{W}, γ_{F}, γ_{nW}, and γ_{nF} of T2 and T2* attenuation may be calculated as 1 in a case in which a time between echoes is short and the influence of T2 and T2* attenuation is small or a case in which high precision is not required.

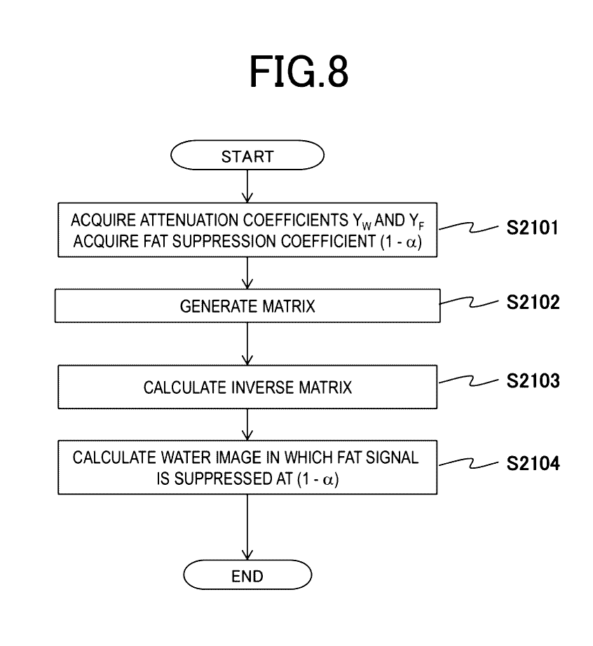

In this case, a matrix may be generated directly without calculating the weight coefficients, an inverse matrix may be calculated, and a water image in which a fat signal is suppressed by a desired ratio may be obtained. The flow of a process by the image processing unit 305 in this case is illustrated in FIG. 8.

(step S2101) The attenuation coefficients of T2 and T2* attenuation between echo signals are acquired from the parameter retention unit 307. The acquired attenuation coefficient for an n-th water signal is referred to as γ_{nW} and the acquired attenuation coefficient for an n-th fat signal is referred to as γ_{nF}. Even in the embodiment, the fat signal suppression ratio (1−α) designated by the user is acquired.

(step S2102) A matrix expressed by Formula (19) below is generated by using the attenuation coefficients γ_{nW} and γ_{nF} and the fat signal suppression ratio (1−α).

$$C = [ \begin{matrix}1 & exp ( i {θ}_{1} ) - α \\ {γ}_{2 W} & {γ}_{2 F} exp ( i {θ}_{2} ) - {γ}_{2 W} α \\ ⋮ & ⋮ \\ {γ}_{nW} & {γ}_{n F} exp ( i {θ}_{2} ) - {γ}_{nW} α\end{matrix} ] \qquad ( 19 )$$

This matrix is generated once in imaging in which one image is obtained.

(step S2103) An inverse matrix C′ of the matrix C generated in step S2102 is calculated. The calculation formula of the matrix is expressed in Formula (20) below.

C′=(C^{H}C)⁻¹C^{H} (20)

In a case in which echo signals are measured at two echo times, the matrix C becomes a square matrix. Accordingly, the inverse matrix C′ may be obtained by Formula (21) below.

C′=C⁻¹ (21)

The inverse matrix is calculated once in one-time imaging.

(step S2104) The water image in which the fat signal remains by α is obtained according to Formula (22) below.

P=C′·S (22)

### Here,

$$[ \begin{matrix}{S}_{1} \\ {S}_{2} \\ ⋮ \\ {S}_{n}\end{matrix} ] = S , [ \begin{matrix}W + α F \\ F\end{matrix} ] = P$$

The component W+αF of the calculated vector P is the water image in which the fat signal remains by α. The calculation of this step is repeatedly performed by the number of image pixels and the number of imaged slices.

As described above, the magnetic resonance imaging apparatus 100 according to the embodiment includes the image processing unit 305 that obtains a water image in which a fat signal is suppressed by a desired ratio by weighting and adding a plurality of images obtained by reconstructing echo signals acquired at echo times with different lengths. The echo times are set such that a phase difference between a water signal and the fat signal included in the image is different in at least two images.

The different echo times are two echo times, the first and second echo times. The phase of the fat signal of the first image reconstructed from the echo signal acquired at the first echo time may be different from the phase of the fat signal of the second image reconstructed from the echo signal acquired at the second echo time. Further, the phases of the water and fat signals may be different in at least one of the first and second images. The water signal and the phase signal of the first image are in the out-phase and the water signal and the fat signal of the second image may be in the in-phase.

The weight coefficient used at the time of the weighting and adding may be decided so that a difference in a signal strength by a difference in the echo time is corrected and the fat signal is suppressed by the desired ratio in the water image. At this time, the image processing unit 305 may decide the weight coefficient to be multiplied to each of the plurality of images using the attenuation correction coefficient to be multiplied to correct the difference in the signal strength or the attenuation coefficient of the signal strength which is a reciprocal of the attenuation correction coefficient and indicates the difference in the signal strength, and the fat suppression coefficient for specifying the ratio by which the fat signal is suppressed.

In this way, according to the embodiment, the coefficients to be multiplied to each of the image data acquired at the TE at which the water and fat signals are in the in-phase and the TE at which the water and fat signals are in the out-phase are decided, and the water image in which the fat signal is suppressed by the desired ratio is obtained by calculation. Accordingly, it is possible to obtain a desired image without once generating the water and fat images in which the water and fat signals are separated. Accordingly, it is possible to obtain the desired image at a high speed without an increase in a use amount of a memory.

In the embodiment, when the coefficient to be multiplied is decided, no fat signal with the out-phase remains in the finally obtained water image in consideration of the influence of T2 and T2* attenuation by the time difference between the TE at which the water and fat signals are in the in-phase and the TE at which the water and fat signals are in the out-phase. That is, the fat signal in the out-phase with respect to the water signal is set to be equal to or less than the fat signal in the in-phase, the weight coefficient is then decided so that the fat signal remains by the desired ratio in the water image, and the weight calculation of the out-phase image and the in-phase image is performed.

Accordingly, the phase of the fat signal remaining in the water image is arranged with the phase of the water signal and the water image in which the fat signal remains by the desired ratio can be obtained without damaging the contrast. That is, the fat suppression ratio and the contrast of the finally obtained water image can be maintained with good quality and the desired image can be obtained with high precision. The slight fat signal remaining in the water image is helpful to comprehend a positional relation between tissues, and thus it is possible to provide an image which is easy to read and in which fat is suppressed.

Further, the parameter for deciding the degree of fat suppression is configured to be set by the user, and thus the operator can freely adjust a fat ratio in accordance with an imaging site or an imaging type. Thus, it is possible to obtain the image with the desired good-quality contrast in each imaging.

Conversely, the database (the fat ratio table 800) in which the ratio by which the fat signal is suppressed is retained in association with at least one of an imaging site or an imaging type may be configured to be included, and thus it is possible to obtain an image with good-quality contrast in which the appropriate fat ratio is set even when the operator is not conscious of the fat ratio.

### REFERENCE SIGNS LIST

100 MRI apparatus
100 magnetic resonance imaging apparatus
101 object
102 static magnetic field generation magnet
103 gradient magnetic field coil
104 irradiation coil
105 reception coil
106 bed
107 gradient magnetic field power source
108 RF transmission unit
109 signal detection unit
110 signal processing unit
111 display unit
112 control unit
113 input unit
200 pulse sequence
201 RF pulse
202 slice encoding gradient magnetic field
203 phase encoding gradient magnetic field
204 frequency encoding gradient magnetic field
205 frequency encoding gradient magnetic field
206 frequency encoding gradient magnetic field
207 frequency encoding gradient magnetic field
211 echo signal
212 echo signal
301 signal reception unit
302 k-space database
303 image conversion unit
304 image database
305 image processing unit
306 image transmission unit
307 parameter retention unit
601 image
602 image
603 image
701 image
702 image
800 fat ratio table
801 imaging site
802 imaging type
810 fat suppression coefficient input region

1. A magnetic resonance imaging apparatus comprising:
an image processing unit that obtains a water image in which a fat signal is suppressed by a desired ratio by weighting and adding a plurality of images obtained by reconstructing echo signals acquired at echo times with different lengths,
wherein the echo times are set such that a phase difference between a water signal and the fat signal included in the image is different in at least two images.

2. The magnetic resonance imaging apparatus according to claim 1,
wherein a weight coefficient used at the time of the weighting and adding is decided so that a difference in a fat signal strength by a difference in the echo time is corrected and the fat signal is suppressed by the desired ratio in the water image.

3. The magnetic resonance imaging apparatus according to claim 2,
wherein the weight coefficient used at the time of the weighting and adding is expressed by a ratio (α) at which the fat signal remains in the water image and an attenuation correction coefficient (β_{F}) for correcting an influence of T2 and T2* attenuation of the fat signal by the difference in the echo time.

4. The magnetic resonance imaging apparatus according to claim 1,
wherein the echo times with the different lengths are two echo times, a first echo time and a second echo time.

5. The magnetic resonance imaging apparatus according to claim 4,
wherein phases of a fat signal of a first image reconstructed from an echo signal acquired at the first echo time and a fat signal of a second image reconstructed from an echo signal acquired at the second echo time are different, and phases of the water signal and the fat signal are different in at least one of the first and second images.

6. The magnetic resonance imaging apparatus according to claim 5,
wherein the water signal and the fat signal of the first image are in an out-phase, and
wherein the water signal and the fat signal of the second image are in an in-phase.

7. The magnetic resonance imaging apparatus according to claim 2,
wherein the image processing unit decides the weight coefficients to be multiplied to each of the plurality of images using an attenuation correction coefficient to be multiplied to correct the difference in the signal strength or an attenuation coefficient indicating the difference in the signal strength and a fat suppression coefficient for specifying a ratio by which the fat signal is suppressed.

8. The magnetic resonance imaging apparatus according to claim 1, further comprising:
an interface that receives designation of a ratio by which the fat signal is suppressed from a user.

9. The magnetic resonance imaging apparatus according to claim 1, further comprising:
a database that maintains a ratio by which the fat signal is suppressed in association with at least one of an imaging site and an imaging type,
wherein the image processing unit acquires the ratio from the database according to an imaging site or an imaging type set by the user.

10. A fat suppression water image calculation method comprising:
a coefficient acquisition step of acquiring an attenuation correction coefficient for correcting a difference in a signal strength by a difference in an echo time between echo signals acquired at a plurality of different echo times or an attenuation coefficient indicating the difference in the signal strength by the difference in the echo time and a fat suppression coefficient for specifying a ratio by which a fat signal is suppressed;
a weight coefficient calculation step of calculating a weight coefficient to be multiplied to a plurality of images reconstructed from the echo signals acquired at the plurality of different echo times from the attenuation correction coefficient or the attenuation coefficient and the fat suppression coefficient; and
a fat suppression water image calculation step of obtaining a water image in which the fat signal is suppressed by the ratio by weighting and adding the plurality of images using the calculated weight coefficient,
wherein the echo times are set such that a phase difference between a water signal and the fat signal included in the image is different in at least two images.
